(19) United States
(12) Patent Application Publication
(10) Pub. No.: US 2007/0253912 A1
(43) Pub. Date: Nov. 1, 2007

(54) PROCESS FOR THE PREPARATION OF MICRONISED COLLAGEN, AND ITS THERAPEUTIC APPLICATIONS

(75) Inventors: DIEGO FURLAN, SAN FELICE (IT); LEONARDO CAPPELLETTI, MONZA (IT)

Correspondence Address: DYKAS, SHAVER & NIPPER, LLP, P.O. BOX 877, BOISE, ID, 83701-0877, US

(73) Assignee: EURORESEARCH SRL, MILAN (IT)

(21) Appl. No.: 11/747,765

(22) Filed: May 11, 2007

Related U.S. Application Data
(63) Continuation-in-part of application No. 10/169,857, filed on Jul. 9, 2002, now abandoned.

(30) Foreign Application Priority Data
Feb. 7, 2001 (EP) PCT/EP01/01283
Feb. 15, 2000 (IT) MI2000A000246

(51) Int. Cl. A61K 38/39; A61K 9/12; C07K 14/78

(52) U.S. Cl. 424/45; 514/12; 530/356

(57) ABSTRACT

Non-denatured type I native collagen extracted from animal tendons is purified and brought into the form of a stable, sterile and meterable powder having a particle size of not more than 20 microns. The powdered collagen so obtained has favourable characteristics of distribution and is particularly efficacious in the more delicate phases of the wound-healing process compared with powdered collagens obtained by known methods.

### CROSS-REFERENCE TO RELATED APPLICATIONS

This application is a continuation-in-part application of co-pending application Ser. No. 10/169,857, filed July 9, 2002, entitled PROCESS FOR THE PREPARATION OF MICRONISED COLLAGEN, AND ITS THERAPEUTIC APPLICATIONS, the disclosure of which is incorporated herein by this reference.

### FIELD OF THE INVENTION

The present invention relates to a process for the preparation of powdered collagen starting from native type I collagen.

Collagen, a polypeptide substance having a molecular weight of approximately 300.000 Daltons is the most abundant fibrous protein in the higher vertebrates because it is the principal constituent of the skin, the connective tissue and the organic material present in the bones and teeth, and represents approximately one third of the total amount of proteins in the human body (Alberts et al “Molecular Biology of the Cell” Garland Publishing Inc., New York and London. 1983).

The production of collagen in the bodies of mammals is preceded by the formation of a larger biosynthetic precursor, precollagen, which is then degraded by specific enzymes to form collagen. Various types of collagen occur naturally and they are all composed of three polypeptide chains which have a constant periodicity and which are arranged in a triple helix; the difference between the various types of collagen is caused by small differences in the primary structure of the chains.

Type I collagen, which is the basic constituent of the skin, bones and tendons, may be regarded as the most abundant of the various types of collagen; it has a 2α1 (I) α2 (I) chain composition where the two α1 chains and the α2 chain are homologous. Moreover type I collagen shows typical polymeric aggregations in fibrils and fibers and this is the situation of its native non-denatured form in nature. Present between the two α1 chains and the α2 chain and between the molecules which give origin to the polymeric aggregations are electrostatic interactions and hydrogen bonds which, together with the presence of hydroxyproline, confer on the molecule characteristics of toughness and strength.

The literature discloses the use of collagen as a stimulating agent in the process of wound-healing by interaction with various growth factors, for its action of capturing fibronectin, as well as the migration and replication of cells which are the consequence thereof (Il collagene nella cicatrizzazione (Collagen in wound-healing) by B. Palmieri, published by Artestampa, January 1990, pages 40-42), and for other actions which have not yet been sufficiently clarified.

U.S. Pat. No. 6,761,908 discloses a denatured collagen preparation for the controlled release of active substances. GB2274458 discloses a denatured collagen which is suitable for use in the clarification of liquids. Denatured collagen preparations are also disclosed by JP08-027035, U.S. Pat. No. 5,356,614 and U.S. Pat. No. 5,196,185. JP10-182499 discloses collagen preparations for the sustained release of ophthalmic active ingredients.

In therapeutics, collagen is currently used as a wound-healing agent in clinical surgery, in the treatment of burns, as a vehicle, in surgical prosthesis (suture threads, gauzes, etc.), as a material for implantation, or as a raw material of creams and ointments in the pharmaceutical and cosmetics sector (Beghè, Mian and Palmieri in Collageno e cicatrizzazione “Realtà e prospettive terapeutiche”(Collagen and wound-healing “Facts and therapeutic perspectives”), Istanbul, 1990; Mian & Mian, Topical collagen and wound Healing, 1992, supplement to vol. XIV; Int.J of tissue reactions, Palmieri, Trabucchi and Zucchelli, Collagene e cicatrizzazione, (Collagen and wound-healing) 1993, Tacchi Ed. Pisa; GELFIX® technical gazette of Euroresearch Srl).

Precisely for its characteristics of toughness and strength, the collagen normally used in pharmaceutical sector is type I collagen.

Collagen is normally obtained in the stable and non-denatured form currently on the market by extraction and purification processes from animal organs, such as, for example, described in JP 2886164. The collagen so obtained is normally a gel which contains from 0.1 to 2.0% of collagen and which, in order then to be used in the various therapeutic applications indicated above, is normally subjected to further conversions; it is, for example, converted by lyophilisation into a pad having a water content of approximately 17%, or, by drying, into a lamina having a water content of approximately 20%.

The powdered collagen currently on the market has, however, disadvantages and defects of not inconsiderable importance because it is available only in a coarse particle size (>500 microns) which does not enable it to adhere to moist surfaces and prevents it from being used in the form of a spray.

However, it will be appreciated that it is important to be able to rely on a collagen which, as far as possible, is non-denatured, anallergic, free from undesired impurities or contaminants and, above all, is in a finely micronised form, that is to say, in order to enable it to be applied to moist and irregular surfaces, such as, for example, the epidermis or damaged tissue.

The object of the present invention is therefore to obtain a product which, while maintaining the typical characteristics of native type I collagen as regards its wound-healing activity, permits easy, simple and rapid application, is easy and practical to use, can be applied to areas of the body which are difficult of access (for example cavities and recesses), and is sterile and structurally homogeneous.

The above has now been obtained by a particular micronisation process which constitutes one of the subjects of the present invention and which enables powdered collagen having a particle size of not more than 20 microns to be obtained.

The micronisation process according to the present invention utilises the normal atomisers currently on the market, such as the rotary cyclone atomisers produced by Niro A/S and described, for example, in United States patents U.S. Pat. No. 5,632,100, U.S. Pat. No. 5,615,493, U.S. Pat. No. 4,490,403, U.S. Pat. No. 4,369,091 and U.S. Pat. No. 3,956,521, which are incorporate herein by reference.

The operation of those atomisers is well known to persons skilled in the art and therefore will not be discussed in detail here; it is, however, important to underline that they basically comprise a cyclone structure where a solution of the product to be micronised is introduced through a rotating nozzle which brings about the nebulisation thereof and is struck by an ascending stream of an inert gas, generally air, heated to a temperature of the order of from 150 to 400° C.

In the present process, however, a 0.1-0.8% by weight/volume aqueous solution of collagen having a pH of from 3.0 to 6.0 is introduced into a normal atomiser and struck by a stream of gas having a temperature substantially lower than those used in the usual micronisation processes; the stream of inert gas, generally air, in fact has a temperature lower than 120° C., preferably of from 70 to 120° C., and even more preferably from 80 to 100° C.

The aqueous collagen solution, generally obtained by diluting a 1.0-2.0% by weight/volume gel of type I native collagen with slightly acidic water, preferably has a final pH of from 4 to 5 and a content of collagen of from 0.3 to 0.5% by weight/volume; the collagen powder is then preferably collected in a closed container which is in a form such that the powder maintains a moisture content of less than 15%.

The product so obtained is a collagen powder having a particle size of from 5 to 30 microns, generally not more than 20 microns and preferably of approximately 18 microns, which maintains intact the quaternary aggregation form (in bundles of fibrils) typical of native collagen; the powder so obtained can then be divided up, placed in suitable containers and sterilized (spray dispensers, sachets, bottles, etc.) according to methods known in the art.

The particle size of not more than 20 microns permits both optimum adhesion of the collagen to the wound surface and its use in metering systems in spray dispensers. This last aspect, in particular, is a very important characteristic of the present invention because the spray formulation permits the production of “multidose” packaging which has the enormous advantage of permitting the discontinuous and repeated use of the product without altering its characteristics and sterility.

On the contrary, because spray-form packaging is possible only for products having a particle size of not more than 20 microns, powdered collagen formulations are currently marketed only in the form of sachets and bottles. Such forms of packaging are of course not very suitable for the therapeutic uses of collagen: sachets, in addition to being difficult to meter, once opened cannot be re-used being no more guaranteed the sterility of the product.

According to the preferred embodiment of the invention, the diluted gel is atomised in an atomiser operating under the following conditions:
temperature of the nozzle: 80-90° C.
pressure of the nebuliser: 1-3 bar
temperature of the gel on entry: 80° C.
temperature of the air: 80° C.
temperature of the product on discharge: 65° C.
moisture content of the product on discharge: 10-15%.

The micronised collagen obtained under the conditions described above has the following characteristics:
particle size: from 5 to 20 microns (98%),
moisture content: not more than 16%.

The micronised collagen can then be packaged in the forms of administration known in the art, generally in combination with the normal excipients and coadjuvants; the preferred formulations are, for example, sachets of from 0.1 to 50 grams, bottles of from 0.5 to 250 grams, spray dispensers of from 10 to 1000 ml; in this last case it is of course necessary to add a suitable propellant gas, generally a preconstituted mixture of n-butane, isobutane and propane gases. The finished products are then subjected to a sterilisation treatment by gamma rays irradiation.

As it shall be appreciated by the following examples, the micronised type I native collagen of the present invention maintains intact the quaternary aggregation form of native collagen and it is thus suitable for the wound-healing; furthermore, it remains stable for a period of at least 60 months at 25° C. with a humidity of 65% and can thus be stored at room temperature without compromising its technical features.

### EXAMPLE 1

500 litres of a 1.2% by w/v collagen solution are diluted with 1500 litres of distilled water (dilution ratio 1:4) in order to obtain 2000 litres at 0.4% by w/v. The pH of the solution is corrected to 4.5±0.5 using dilute acetic acid.

The solution is then introduced into an atomiser under the following operating conditions:
air and nozzle temperature: 80-85° C.
temperature on entry: 80° C.
temperature on discharge: 65° C.
pressure of the nebuliser: 2 bar
capacity of the feed pump: 40 l/hour.

At the end of the operation, approximately 8.0 kilograms of powdered collagen having the desired particle size (98%<20 microns) and moisture content (K.F. 14%) are collected.

### EXAMPLE 2

The micronised collagen obtained in the manner described in Example 1 is packaged automatically in bottles of neutral glass having a cap and an under-cap of non-toxic plastics material; using doses of 1, 2 and 5 grams per bottle.

The bottles so obtained are subjected to treatment with gamma rays irradiation at a dose of 25 kilograys in order to obtain a powder completely free from micro-organisms.

### EXAMPLE 3

The micronised collagen obtained in the manner described in Example 1 is packaged automatically in aluminium spray dispensers having an internal lining of epoxy resin. 50 and 75 ml spray dispensers containing, respectively, 1 and 2 grams of micronised collagen are used. The spray dispensers are then equipped with delivery valves and the propellant composed of a preconstituted mixture of n-butane, isobutane and propane (95:2:3) is then introduced at a pressure of approximately 1.3 bar.

The spray dispensers so obtained are subjected to treatment with gamma rays irradiation at a dose of 25 kilograys in order to obtain a powder completely free from micro-organisms.

### EXAMPLE 4

The preservation of the native structure of the micronised collagen obtained in the manner described in Example 1 (six different lots) has been confirmed by the determination of water-soluble peptides, i.e. hydrolyzed collagen products originated by the extraction, micronisation and sterilization process. Such test was conducted in comparison with standard type I native collagens (SIGMA 129F8130, SIGMA128F81451, CALBIOCHEM 014491). The results are reported in table 1.

TABLE 1

| Miconised | | | |
|---|---|---|---|
| collagen of | | | |
| the invention | % of water- | | % of water-soluble |
| lot n. | soluble peptides | Standard lot n. | peptides |
| 2389 | 10.60 | S-129F8130 | 10.40 |
| 2689 | 12.00 | S-128F81451 | 6.60 |
| 3389 | 9.00 | C-014491 | 7.80 |
| 3889 | 11.00 | | |
| 4089 | 7.00 | | |
| 4389 | 8.00 | | |
| Mean ± SD | 9.60 ± 1.90 | Mean ± SD | 8.30 ± 1.94 |

The statistical evaluation performed with Student's “t” test confirmed that the groups of tested values were statistically comparable. The obtained results also demonstrates that the micronised type I native collagen maintains intact the quaternary aggregation form of native collagen.

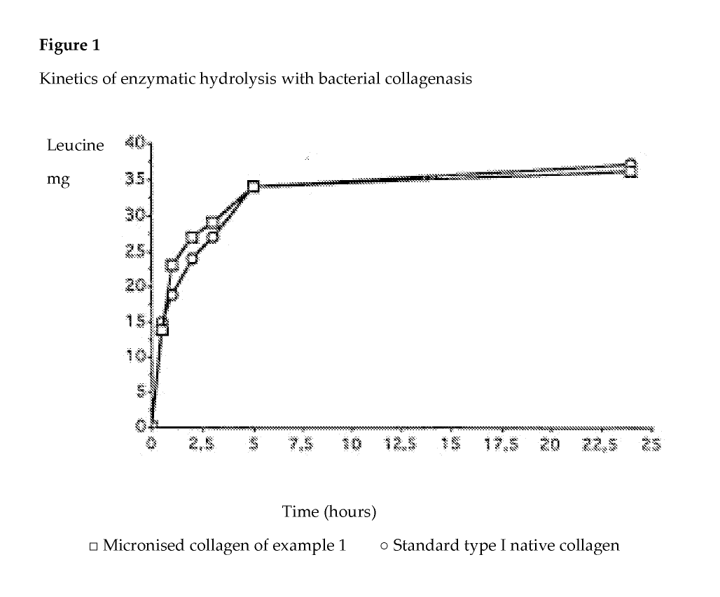

Furthermore, since collagen may also be “damaged” by manufacturing processes, an additional test has been performed in order to measure such a “damage” by means of enzymatic hydrolysis of bacterial collagenasis. By a long term evaluation (24 hours) it is in fact possible to describe a kinetics of such enzymatic hydrolysis in comparison with standard type I native collagen. The results of such a comparative test, which are reported in FIG. 1, provide an additional confirmation that the micronised type I native collagen of the present invention maintains intact the quaternary aggregation form of native collagen.

### EXAMPLE 5

Samples belonging to lots 0010S, 00201S and 00301S of the micronised collagen obtained in the manner described in Example 1 have been maintained in a thermoregulated thermostat at 25° C. with a humidity of 60% for 60 months. The following analysis have been performed on the samples:
a—Appearance
b—Total nitrogen
c—Hydroxyproline
d—Free hydroxyproline

The results are summarized in Tables 2, 3 and 4

TABLE 2
(lot 00101S)

| | | 3 | 6 | 12 | 24 | 36 | 48 | 60 |
|---|---|---|---|---|---|---|---|---|
| Features | time 0 | months | months | months | months | months | months | months |
| Appearance | good* | good* | good* | good* | good* | good* | good* | good* |
| Total nitrogen | 3.60% | 3.55% | 3.57% | 3.53% | 3.97% | 3.88% | 3.72% | 3.64% |
| | s.s. | s.s. | s.s. | s.s. | s.s. | s.s. | s.s. | s.s. |
| Hydroxyproline | 2.84% | 2.74% | 2.75% | 2.63% | 2.90% | 2.88% | 2.62% | 2.73% |
| | s.s. | s.s. | s.s. | s.s. | s.s. | s.s. | s.s. | s.s. |
| Free | 0.10% | 0.10% | 0.09% | 0.11% | 0.11% | 0.12% | 0.10% | 0.12% |
| hydroxyproline | | | | | | | | |

*White ivory microcrystalline powder with typical smell

TABLE 3
(lot 00201S)

| | | 3 | 6 | 12 | 24 | 36 | 48 | 60 |
|---|---|---|---|---|---|---|---|---|
| Features | time 0 | months | months | months | months | months | months | months |
| Appearance | good* | good* | good* | good* | good* | good* | good* | good* |
| Total nitrogen | 3.54% | 3.62% | 3.64% | 3.51% | 3.54% | 3.72% | 3.80% | 3.75% |
| | s.s. | s.s. | s.s. | s.s. | s.s. | s.s. | s.s. | s.s. |
| Hydroxyproline | 2.77% | 2.82% | 2.62% | 2.78% | 2.90% | 2.92% | 2.88% | 2.78% |
| | s.s. | s.s. | s.s. | s.s. | s.s. | s.s. | s.s. | s.s. |
| Free | 0.12% | 0.10% | 0.08% | 0.16% | 0.10% | 0.12% | 0.10% | 0.12% |
| hydroxyproline | | | | | | | | |

*White ivory microcrystalline powder with typical smell

TABLE 4
(lot 00301S)

| | | 3 | 6 | 12 | 24 | 36 | 48 | 60 |
|---|---|---|---|---|---|---|---|---|
| Features | time 0 | months | months | months | months | months | months | months |
| Appearance | good* | good* | good* | good* | good* | good* | good* | good* |
| Total nitrogen | 3.82% | 3.77% | 3.54% | 3.55% | 3.71% | 3.64% | 3.61% | 3.70% |
| | s.s. | s.s. | s.s. | s.s. | s.s. | s.s. | s.s. | s.s. |
| Hydroxyproline | 2.86% | 2.80% | 2.79% | 2.86% | 2.70% | 2.85% | 2.78% | 2.88% |
| | s.s. | s.s. | s.s. | s.s. | s.s. | s.s. | s.s. | S.S. |
| Free | 0.11% | 0.10% | 0.08% | 0.16% | 0.11% | 0.10% | 0.11% | 0.15% |
| hydroxyproline | | | | | | | | |

*White ivory microcrystalline powder with typical smell

The above data demonstrates that the micronised type I native collagen of the present invention remains stable for a period of at least 60 months at 25° C. with a humidity of 65%.

1. Non-denatured micronised type I native collagen, which maintains intact the quaternary aggregation form of native collagen, obtainable by means of a process in which a 0.1-0.8% by weight/volume aqueous collagen solution having a pH of from 3.0 to 6.0 is brought into contact with a stream of gas in an atomiser, characterised in that the stream of gas has a temperature of less than 120° C. and in that the starting collagen consists of type I native collagen.

2. Non-denatured micronised type I native collagen according to claim 1, characterised in that the stream of gas has a temperature of from 70 to 120° C.

3. Non-denatured micronised type I native collagen according to claim 2, characterised in that the stream of gas has a temperature of from 80 to 100° C.

4. Non-denatured micronised type I native collagen according to claim 1, characterised in that the aqueous collagen solution is a 0.3 to 0.5% by weight/volume solution.

5. Non-denatured micronised type I native collagen according to claim 1, characterised in that the aqueous collagen solution has a pH of from 4.0 to 5.0.

6. Non-denatured micronised type I native collagen according to claim 1, characterised in that the stream of gas is constituted by air.

7. Non-denatured micronised type I native collagen according to claim 1, characterised in that the atomiser is a rotary cyclone atomiser.

8. Non-denatured micronised collagen according to claim 1, characterised in that it has a particle size of not more than 20 microns.

9. Non-denatured micronised collagen according to claim 8, characterised in that it has a particle size of approximately 18 microns.

10. A pharmaceutical composition, characterized by consisting of non-denatured micronised type I native collagen according to claim 1 and by optionally containing excipients and coadjuvants.

11. A composition according to claim 10, characterized by being in the form of a spray and by further containing a propellant gas.

12. A composition according to claim 11, characterized in that said propellant gas is selected from n-butane, isobutane and/or propane.

13. A wound-healing method which comprises applying a composition according to claim 10 to the surface of the body of a patient in need of such a treatment.
